(19) United States
(12) Plant Patent Application Publication
(10) Pub. No.: US 2008/0134394 P1
(43) Pub. Date: Jun. 5, 2008

(54) Geranium plant named 'Zonadared'

(75) Inventor: Theodorus Cornelis Maria van Kleinwee, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/607,149

(22) Filed: Dec. 1, 2006

(51) Int. Cl. A01H 5/00

(52) U.S. Cl. PLT/330

(57) ABSTRACT

A new Geranium plant, particularly distinguished by the deep scarlet red flower color, green foliage with strong zone, and a very nice round plant structure with good branching.

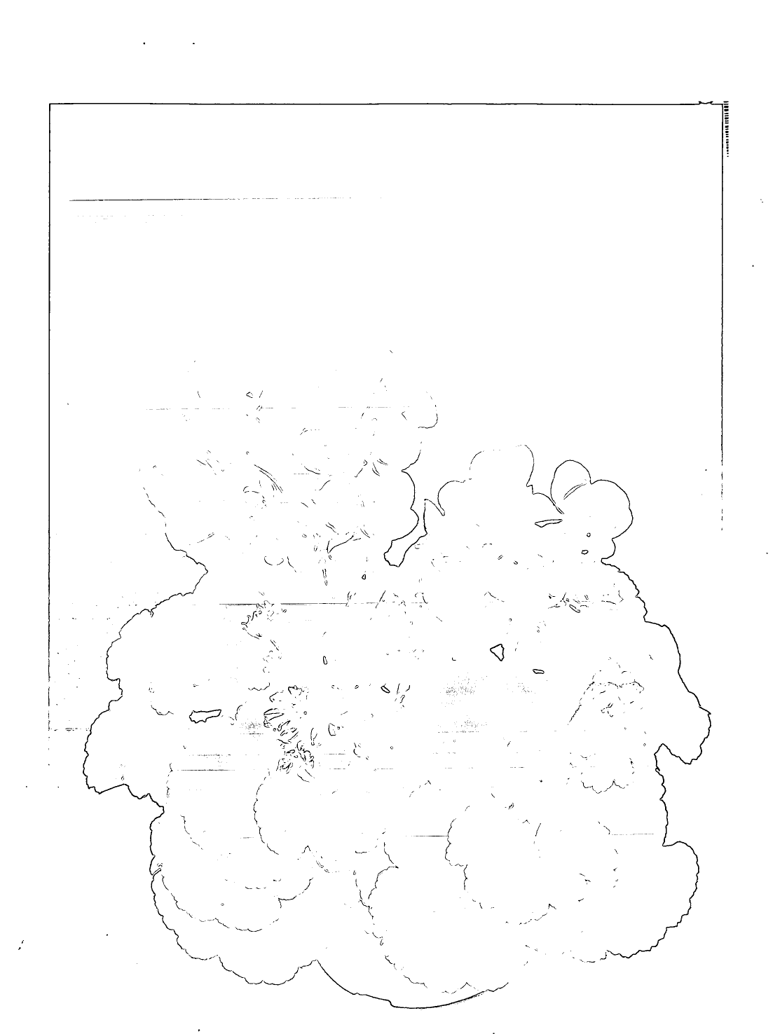

### LATIN NAME OF THE GENUS AND SPECIES OF THE PLANT CLAIMED

Pelargonium zonale (L.) L'Hérit. ex Ait

### VARIETAL DENOMINATION

‘Zonadared’

### BACKGROUND OF THE NEW PLANT

The present invention comprises a new Geranium, botanically known as Pelargonium zonale (L.) L'Hérit. ex Ait, and hereinafter referred to by the variety name ‘Zonadared.’

‘Zonadared’ is a product of a planned breeding program. The new cultivar ‘Zonadared’ has large, semi-double flowers, is relatively vigorous, but with a well-branched growth habit, and has good heat resistance.

‘Zonadared’ originates from a hybridization in a controlled breeding program in Enkhuizen, The Netherlands. The female parent was an unpatented hybrid seedling identified as ‘T042’ with deep scarlet color, nice zone on the foliage, and a long flower stem. The male parent of ‘Zonadared’ was an unpatented hybrid seedling identified as ‘J2242-1’ with scarlet red color, big umbel, and dark foliage. ‘Zonadared’ was selected as one flowering plant within the progeny of the stated cross in 2000 in a controlled environment in Enkhuizen, The Netherlands.

The first act of asexual reproduction of ‘Zonadared’ was accomplished when vegetative cuttings were taken from the initial selection in the Fall of 2000 in a controlled environment in Enkhuizen, the Netherlands.

Horticultural examination of plants grown from cuttings of the plant initiated in April of 2001 in Enkhuizen, The Netherlands, and continuing thereafter, has demonstrated that the combination of characteristics as herein disclosed for ‘Zonadared’ are firmly fixed and are retained through successive generations of asexual reproduction.

‘Zonadared’ has not been observed under all possible environmental conditions. The phenotype may vary significantly with variations in environment such as temperature, light intensity and day length.

### BRIEF DESCRIPTION OF THE NEW VARIETY

The accompanying photographic drawing shows typical flower and foliage characteristics of ‘Zonadared’ with colors being as true as possible with an illustration of this type. The photographic drawing shows a flowering potted plant of the new variety.

### DETAILED BOTANICAL DESCRIPTION

The measurements were taken in Enkhuizen, The Netherlands, in August 2005 on plants that were growing in window boxes. Culture of these plants started mid February, 2005 in the greenhouse; plants were transferred to window boxes mid May, 2005.

### BRIEF SUMMARY OF INVENTION

The following observations, measurements, and comparisons describe plants grown outside in Enkhuizen, The Netherlands. The following traits have been repeatedly observed and are determined to be basic characteristics of the new variety. The combination of these characteristics distinguishes this Geranium as a new and distinct variety.

TABLE 1
DIFFERENCES BETWEEN THE NEW VARIETY
‘ZONADARED’ AND A SIMILAR VARIETY

| | | ‘Americana |
|---|---|---|
| | | Dark Red’ |
| | | (U.S. Plant Patent |
| | ‘Zonadared’ | No. 9,307) |
| Leaf zoning | Strong zoning | Hardly any zoning |
| Leaf color (upper side) | 137A | 137B |
| Flower color | Top 44B, fading | 46B |
| (upper side of upper petal) | towards middle to 52A | |

| | |
|---|---|
| PLANT | |
| Plant height (cm): | 27 |
| Plant height (inflorescence included) (cm): | 38 |
| Plant width (cm): | 50 |
| Plant shape: | Round |
| FOLIAGE | |
| Length (cm): | 6.5 |
| Width (cm): | 10 |
| Shape: | Reniform |
| Base shape: | Cordate |
| Base size (cm): | 5 |
| Apex shape: | Very weak lobes |
| Apex size (cm): | 6 |
| Color of upper surface: | 137A |
| Color of lower surface: | 146A |
| Color of zone: | 187A |
| Color of veins: | 146C |
| Margin: | Bicrenate |
| Petioles length (cm): | 5-8 |
| Diameter of petiole (mm): | 3 |
| Leaves smooth and hairy: | Only at nerves |
| Texture of leaves: | Upper surface smooth |
| Veins: | Palmate |
| STEM: | |
| Length of internodes (mm): | 10-60 |
| Color of stem: | 146B |
| Diameter (mm): | 9 |
| INFLORESCENCE | |
| Shape: | Round outline |
| Diameter (cm): | 12 |
| Umbel depth (cm): | 6 |
| Length of peduncle (cm): | 10-20 |
| Peduncle diameter (mm): | 3-5 |
| Color of peduncle: | 146B |
| Length of pedicel (cm): | 2-2.5 |
| Color of pedicel: | 144A, with 183B spread over |
| | pedicel |
| Number of flowers per inflorescence: | 70 |
| COROLLA | |
| Form: | Semi double |
| Shape: | Round outline, with the upper |
| | petals about the same size |
| | as the lower petals, cup |
| | shaped |
| Length of flower (cm): | 4.8 |
| Width of flower (cm): | 4.5 |
| Number of petals: | 8-10 |
| Length of petals (upper/lower) (mm): | Upper 28 |
| | Lower 27 |
| Width of petals (upper/lower) (mm): | Upper 18 |
| | Lower 20 |
| Shape petals: | Obovate, base acute, upper |
| | end is truncate or rounded, |
| | margin is entire |
| Color upper side of upper petals: | Top 44B, fading towards |
| | middle to 52A |
| Color upper side of lower petals: | Top 44B, fading towards |
| | middle to 52A |
| Color lower side of upper petals: | 44C |
| Color lower side of lower petals: | 44C |
| Number of petaloids: | 2-3; often very big, almost |
| | size of petals |
| Color petaloids: | 44B |
| Fading color when flower starts aging: | No |
| Duration of flowering: | Continuous flowering |
| | throughout the Summer |
| BUD (just before opening) | |
| Shape: | Narrow elliptic |
| Shape base of bud: | Truncate |
| Shape top of bud: | Acuminate |
| Sepals hairy and smooth: | Yes |
| Length (mm): | 13 |
| Width (mm): | 6 |
| Color: | 146B |
| Number of sepals: | 5 |
| Color of sepals: | 143C |
| Length of sepals (mm): | 7 |
| Width of sepals (mm): | 3 |
| REPRODUCTIVE ORGANS | |
| Number of lobes of stigma: | 5 |
| Color stigma: | 42A |
| Length of style (mm): | 4 |
| Color of style: | 43D |
| Number of anthers: | 7-9 |
| Length of anthers (mm): | 3 |
| Shape of anthers: | Ovate |
| Length filaments (mm): | 7 |
| Color of filaments: | 155D, Top 43B |
| Pollen present: | Yes |
| Color of pollen: | 32A |
| Fertility/seed set: | No |

Disease/pest resistance: No disease/pest resistance has been observed to date.

What is claimed is:

1. A new and distinct variety of Geranium plant, substantially as illustrated and described herein.
